(19) United States
(12) Patent Application Publication
(10) Pub. No.: US 2012/0123546 A1
(43) Pub. Date: May 17, 2012

(54) IMPLANT APPARATUS FOR SPINAL FUSION

(75) Inventor: Mark Patrick Medina, Torrance, CA (US)

(73) Assignee: MEDevice IP Holdings, LLC.

(21) Appl. No.: 13/186,189

(22) Filed: Jul. 19, 2011

Related U.S. Application Data
(60) Provisional application No. 61/413,511, filed on Nov. 15, 2010.
(60) Provisional application No. 61/495,435, filed on Jun. 10, 2011.
(60) Provisional application No. 61/508,759, filed on Jul. 18, 2011.

(51) Int. Cl. A61F 2/44

(52) U.S. Cl. 623/17.16

(57) ABSTRACT

An implant apparatus and a method for spinal fusion from oblique, lateral, ALIF, PLIF, and TLIF approach are disclosed. The apparatus can be configured to include an expandable implant cage and an inserter. The cage can be inserted between endplates of upper and lower vertebra using lateral, ALIF, PLIF, and TLIF approach. The cage generally includes a male and female screw configuration and a cage expansion mechanism. The inserter inserts the cage in a spinal disc space and tightens the male and female screw arrangement. Once the cage is inserted to the desired position, viewed by X-ray you will begin to tighten the male portion of the screw in the device, and continue to tighten until final deployment of cage has been achieved. This provides a much greater footprint that allows the device to reach the cortical ring or apophyseal ring of the vertebral body. Tightening of male and female screw arrangement operates the cage expansion mechanism to expand the cage size. The cage can be inserted through a smaller surgical opening and then expanded to a full size assembly between the vertebrae.

### CROSS-REFERENCE TO PROVISIONAL PATENT APPLICATIONS

This patent application claims the benefit under 35 U.S.C. §119(e) of U.S. Provisional Application Ser. No. 61/413,511 entitled, “Implant Apparatus for Spinal Fusion,” which was filed on Nov. 15, 2010 and is incorporated herein by reference in its entirety. This patent application further claims the benefit under 35 U.S.C. §119(e) of U.S. Provisional Application Ser. No. 61/495,435 entitled, “Implant Apparatus for Spinal Fusion,” which was filed on Jun. 10, 2011 and is incorporated herein by reference in its entirety. This patent application also claims the benefit under 35 U.S.C. §119(e) of U.S. Provisional Application Ser. No. 61/508,759 entitled, “Implant Apparatus for Spinal Fusion,” which was filed on Jul. 18, 2011 and is incorporated herein by reference in its entirety.

### TECHNICAL FIELD

Embodiments are generally related to spinal implants. Embodiments also relate to the field of vertebral body spacers. Embodiments additionally relate to implanting techniques and surgical devices and component devices for spinal fusion.

### BACKGROUND OF THE INVENTION

In some instances, an intervertebral disc that becomes degenerated may need to be partially or fully removed from a spinal column. Intervertebral discs can degenerate due to various causes such as, for example, trauma, disease, or aging. Removal or partial removal of an intervertebral disc destabilizes the spinal column. A spinal implant may thus be inserted into a disc space created by the removal or partial removal of an intervertebral disc. The spinal implant may maintain the height of the spine and restore stability to the spine. Bone then grows from the adjacent vertebrae into the spinal implant. The bone growth fuses the adjacent vertebrae.

A spinal implant can be inserted utilizing an anterior, transforaminal, oblique, posterior or lateral spinal approach. For an anterior approach, extensive vessel retraction is often required and many vertebral levels are not readily accessible from this approach. Another approach is a posterior approach. This approach typically requires that both sides of the disc space on either side of the spinal cord be surgically exposed, which may require a substantial incision or multiple access locations, as well as extensive retraction of the spinal cord.

Yet another approach is a posterior-lateral approach to the disc space. The posterior-lateral approach is employed in a posterior lumbar interbody fusion (PLIF) or transforaminal lumber interbody fusion (TLIF) procedure, which may be performed as an open technique, which requires making a larger incision along the middle of the back. Through this incision, the surgeon then cuts away, or retracts, spinal muscles and tissue to access the vertebrae and disc space. The TLIF procedure may also be performed as a minimally invasive or as an extreme lateral interbody fusion procedure that involves a retroperitoneal transpoas approach to the lumbar spine as an alternative to “open” fusion surgery. In the minimally invasive procedure, the surgeon employs much smaller incisions, avoids disrupting major muscles and tissues in the back, and reduces the amount of muscle and tissue that is cut or retracted.

Anterior Lumbar Interbody Fusion (ALIF) using threaded devices such as cages and bone dowels have been in use for over ten years. Initially, threaded cages or dowels were expected to act as a stand-alone device that would promote fusion and maintain disc height without the need for posterior surgery and instrumentation of the spine. In spite of fusion rates better than 90 percent for single level fusion and 65 percent for two-level fusion, significant subsidence has been observed on follow-up X-rays at varying times following the procedure. This subsidence, or slow insinuation of the threaded devices into the vertebral bodies, has resulted in lost disc height, which in some patients has resulted in the failure to fuse and the recurrence of often very painful symptoms.

The implants may be constructed of any biocompatible material sufficiently strong to maintain spinal distraction including, but not limited to, bone, metals, ceramics and/or polymers. Implants may be packed with bone graft or a synthetic bone graft substitute to facilitate spinal fusion. Implants may have a variety of shapes, which include, but are not limited to, threaded cylinders, unthreaded cylinders, and parallelepipeds.

A protective sleeve can be used during preparation and insertion of a spinal implant. The protective sleeve serves to protect abdominal organs, blood vessels, and other tissue during a spinal implant procedure using an anterior approach. The sleeve typically extends above the surgical opening during use. The sleeve maintains distraction of the vertebrae. Also, the sleeve serves as an alignment guide for tool and implant insertion during the surgical procedure. Protective sleeves can also be used during a spinal fusion procedure using a posterior or lateral approach.

Typically, most surgical corrections of a disc space include at least a partial discectomy, which is followed by restoration of normal disc space height and, in some instances, fusion of the adjacent vertebral bodies. Restoration of normal disc space height generally involves the implantation of a spacer and fusion typically involves inclusion of bone graft or bone graft substitute material into the intervertebral disc space to create bony fusion. Fusion rods may also be employed. Some implants further provide artificial dynamics to the spine. Such techniques for achieving interbody fusion or for providing artificial disc functions are well known.

The inter-vertebral spacing (i.e., between neighboring vertebrae) in a healthy spine can be maintained via a compressible and somewhat elastic disc. The disc serves to allow the spine to move about the various axes of rotation and through the various arcs and movements required for normal mobility. The elasticity of the disc maintains spacing between the vertebrae, allowing room or clearance for compression of neighboring vertebrae, during flexion and lateral bending of the spine. In addition, the disc allows relative rotation about the vertical axis of neighboring vertebrae, allowing twisting of the shoulders relative to the hips and pelvis. Clearance between neighboring vertebrae maintained by a healthy disc is also important to allow nerves from the spinal chord to extend out of the spine, between neighboring vertebrae, without being squeezed or impinged by the vertebrae.

In situations (based upon injury or otherwise) where a disc is not functioning properly, the inter-vertebral disc tends to compress, and in doing so pressure is exerted on nerves extending from the spinal cord by this reduced inter-vertebral spacing. Various other types of nerve problems may be experienced in the spine such as exiting nerve root compression in neural foramen, and passing nerve root compression. A few medical procedures have been devised to alleviate such nerve compression and the pain that results from nerve pressure. Many of these procedures revolve around attempts to prevent the vertebrae from moving too close to each other by surgically removing an improperly functioning disc and replacing it with a lumber interbody fusion (LIF) device. Although prior interbody devices, including LIF cage devices, may be effective at improving patient condition, the vertebrae of the spine, body organs, the spinal cord, other nerves, and other adjacent bodily structures make obtaining surgical access to the location between the vertebrae where the LIF cage is to be installed difficult.

In case of lateral approach, it would be desirable to reduce the size of the LIF/VBR cage to minimize the size for the required surgical opening for installation of the LIF/VBR cage, while maintaining high strength, durability, and reliability of the LIF/VBR cage device. Instruments and lateral implants are not necessarily suited to efficiently distract the disc space without damaging the adjacent endplates. In an effort to address the foregoing difficulties, it is believed that the implant device for spinal fusion from lateral approach, as discussed herein, can address many of the problems with traditional lateral implants.

### BRIEF SUMMARY

The following summary is provided to facilitate an understanding of some of the innovative features unique to the disclosed embodiment and is not intended to be a full description. A full appreciation of the various aspects of the embodiments disclosed herein can be gained by taking the entire specification, claims, drawings, and abstract as a whole.

It is, therefore, one aspect of the disclosed embodiments to provide for spinal implants.

It is another aspect of the disclosed embodiments to provide for vertebral body spacers.

It is yet another aspect of the disclosed embodiments to provide for implanting techniques and device for spinal fusion from lateral approach.

The aforementioned aspects and other objectives and advantages can now be achieved as described herein. An implant apparatus and a method for spinal fusion from oblique, lateral, ALIF, PLIF, and TLIF approach are disclosed. The apparatus can be configured to include an expandable implant cage and an inserter. The cage can be inserted between endplates of upper and lower vertebra using an oblique, lateral, ALIF, PLIF, and/or TLIF approach. The cage generally includes a male and female screw configuration and a cage expansion mechanism. The inserter inserts the cage in a spinal disc space and tightens the male and female screw arrangement. Once the cage is inserted to the desired position, viewed by X-ray you will begin to tighten the male portion of the screw in the device, and continue to tighten until final deployment of the cage has been achieved. This provides a much greater footprint that allows the device to reach the cortical ring or apophyseal ring of the vertebral body. Tightening of male and female screw arrangement operates the cage expansion mechanism to expand the cage size. The cage can be inserted through a smaller surgical opening and then expanded to a full size assembly between the vertebrae.

### BRIEF DESCRIPTION OF THE DRAWINGS

The accompanying figures, in which like reference numerals refer to identical or functionally similar elements throughout the separate views and which are incorporated in and form a part of the specification, further illustrate the disclosed embodiments and, together with the detailed description of the invention, serve to explain the principles of the disclosed embodiments.

FIG. 1 illustrates a top view of an implant cage utilized for spinal fusion, in accordance with the disclosed embodiments;

FIG. 2 illustrates a perspective view of the implant cage of FIG. 1, in accordance with the disclosed embodiments;

FIG. 3 illustrates a perspective view of a male and female screw arrangement of FIG. 1, in accordance with the disclosed embodiments;

FIG. 4 illustrates a perspective view of an inserter utilized in implantation process, in accordance with disclosed embodiments;

FIG. 5 illustrates a perspective view of the implant device including the implant cage of FIG. 1 and the inserter of FIG. 4, in accordance with the disclosed embodiments;

FIG. 6 illustrates a perspective view of an implant cage, in accordance with an alternative embodiment;

FIG. 7 illustrates a top view of the implant cage of FIG. 6, in accordance with an alternative embodiment;

FIG. 8 illustrates a perspective view of the implant cage of FIG. 6 after expansion, in accordance with an alternative embodiment;

FIG. 9 illustrates a perspective view of a vertebral endplate with the implant cage of FIG. 8, in accordance with an alternative embodiment;

FIG. 10 illustrates a perspective view of an implant cage, in accordance with an alternative embodiment:

FIG. 11 illustrates a perspective view of a vertebral endplate with the implant cage of FIG. 10, in accordance with an alternative embodiment;

FIG. 12 illustrates a high level flow chart depicting implantation process for spinal fusion from lateral approach, in accordance with the disclosed embodiments;

FIG. 13 illustrates a perspective view of an implant apparatus for spinal fusion, in accordance with another embodiment;

FIG. 14 illustrates a left side view, a front view, a right side view, a bottom view, and a top view of the implant cage of the apparatus depicted in FIG. 13;

FIG. 15 illustrates a perspective view of an implant apparatus for spinal fusion, in accordance with another embodiment;

FIG. 16 illustrates a left side view, a front view, a right side view, a bottom view, and a top view of the implant cage of the apparatus depicted in FIG. 15; and

FIG. 17 illustrates a perspective view of an inserter device that can be utilized in accordance with the disclosed embodiments.

### DETAILED DESCRIPTION

The particular values and configurations discussed in these non-limiting examples can be varied and are cited merely to illustrate at least one embodiment and are not intended to limit the scope thereof.

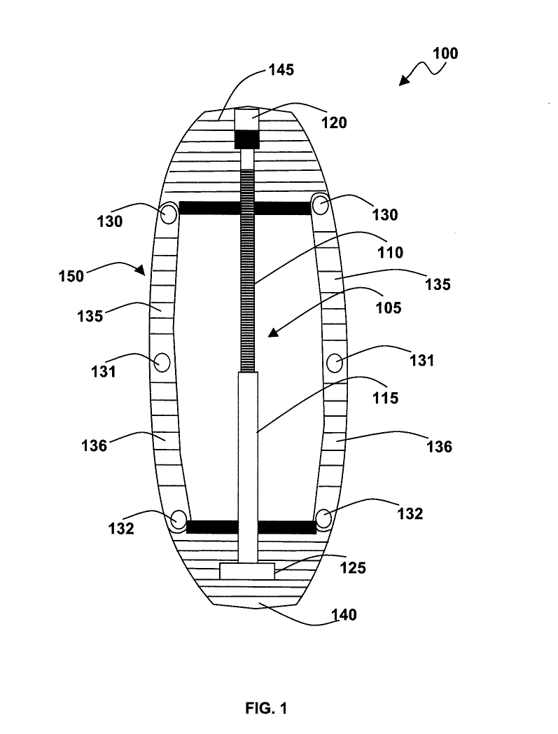

FIG. 1 illustrates a top view of an implant cage apparatus 100 utilized for spinal fusion, in accordance with the disclosed embodiments. Note that as utilized herein the term “spinal fusion” can include, for example, lumbar fusion and other procedures. The cage apparatus 100 includes a male and female screw arrangement 105, and a cage expansion mechanism 150. A head 125 of a male screw 115 and a head 120 of a female screw 110 are positioned on the front side 140 and back side 145 of the cage apparatus 100 respectively. The cage expansion mechanism 150 includes pins 130, 131, and 132, and hinges 135 and 136. The hinges 135 and 136 are generally connected by a common pin 131.

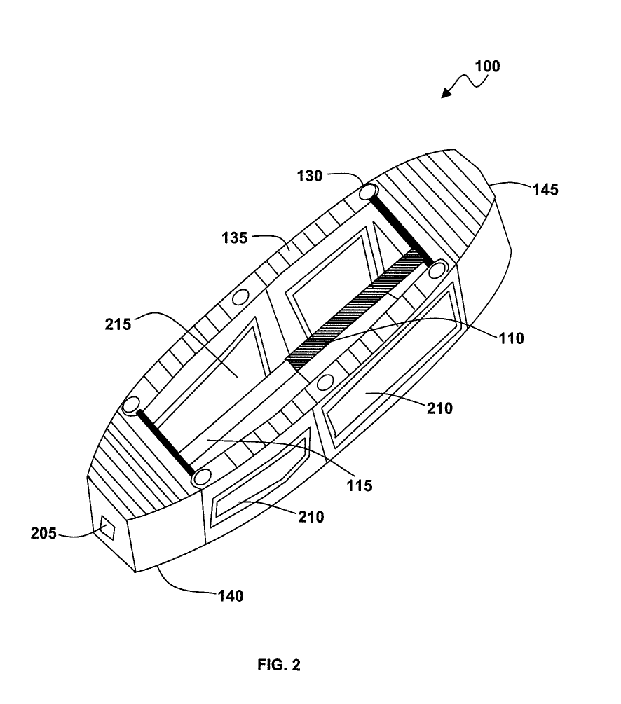

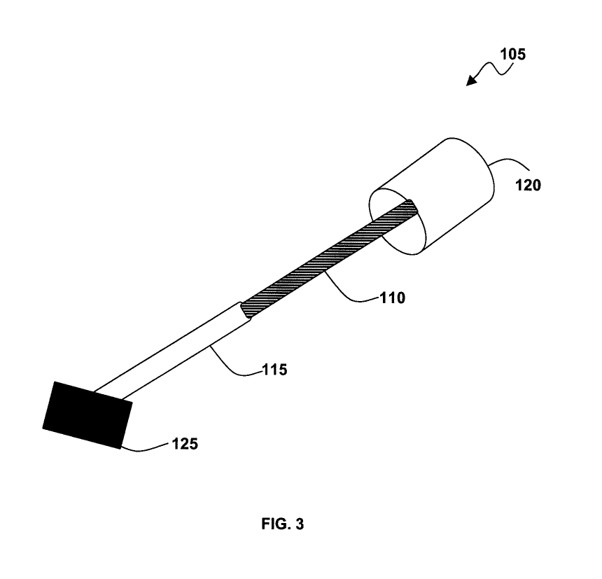

FIG. 2 illustrates a perspective view of the implant cage apparatus 100 of FIG. 1, in accordance with the disclosed embodiments. An aperture 210 allows the healing material to flow in and out of a cavity 215. The cage apparatus 100 can be inserted into the spinal disc space through a port 205 on the front side 140. FIG. 3 illustrates a perspective view of the male and female screw arrangement 105 utilized in FIG. 1, in accordance with the disclosed embodiments. When the male and female screw arrangement 105 is tightened by an inserter (not shown), the cage expansion mechanism 150 expands the cage apparatus 100 and increases it size.

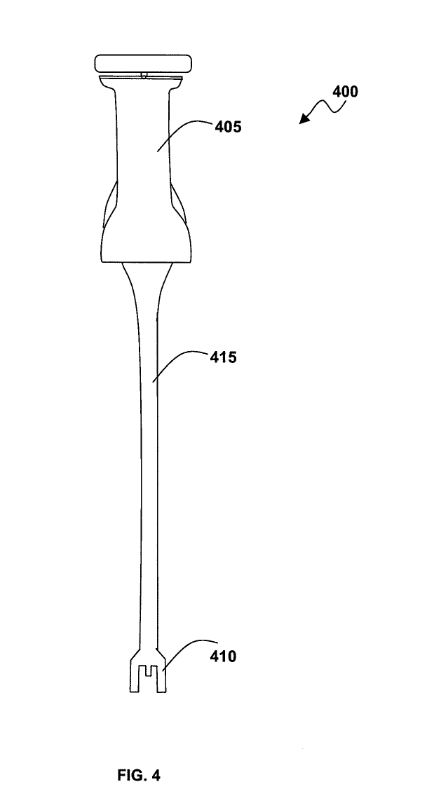

FIG. 4 illustrates a perspective view of an inserter 400 that can be utilized in a spinal implantation process, in accordance with disclosed embodiment. The inserter includes a handle 405, a coupling arrangement 410, and a shaft 415. The inserter 400 is utilized for inserting the cage into spinal disc space (not shown). Inserter 400 is also utilized for tightening the male and female screw arrangement 105. Tightening of the male and female screw arrangement 105 expands the size of the cage apparatus 100.

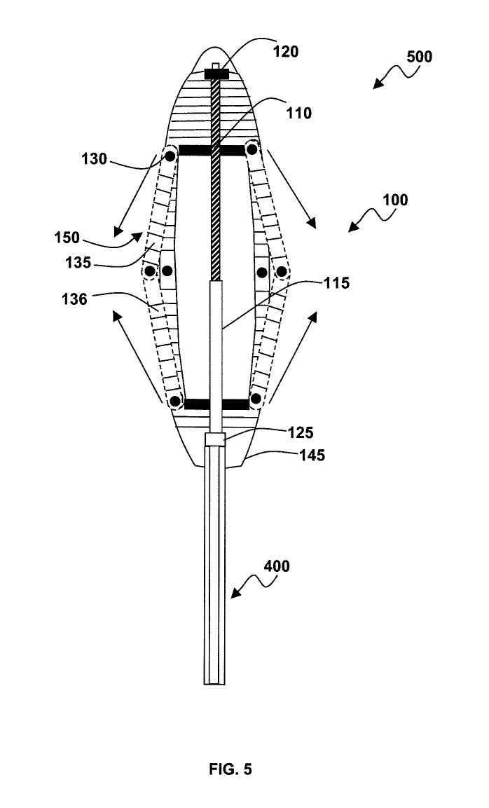

FIG. 5 illustrates a perspective view of the implant device 500 after expansion by utilizing the inserter 400 of FIG. 4, in accordance with the disclosed embodiments. The implant device 500 includes the cage apparatus 100 and inserter 400. Note that the hinges 135 and 136 can be configured to stretch generally outside the cage and thus increase the size of the cage apparatus 100.

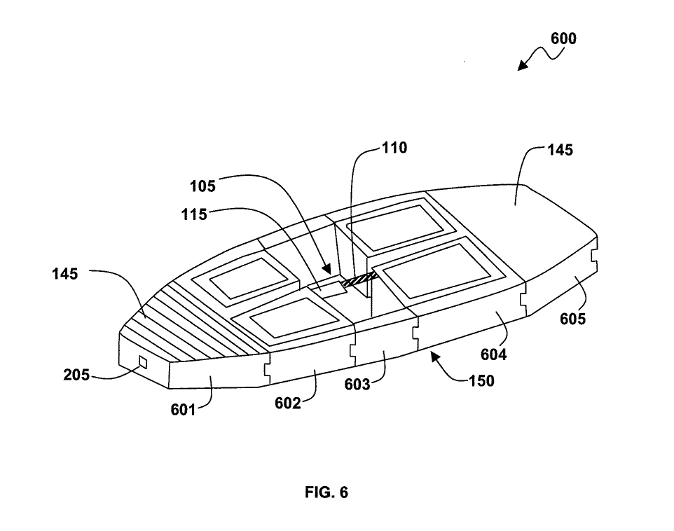

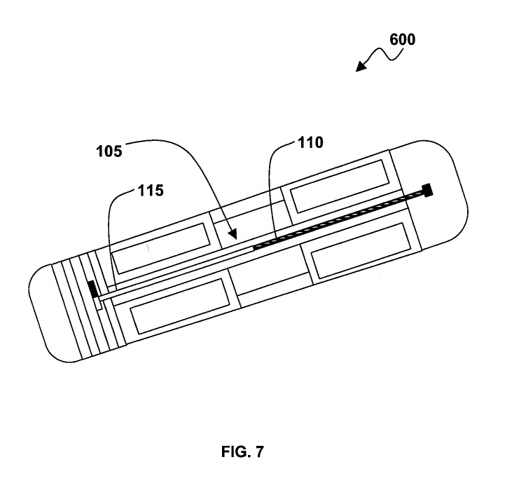

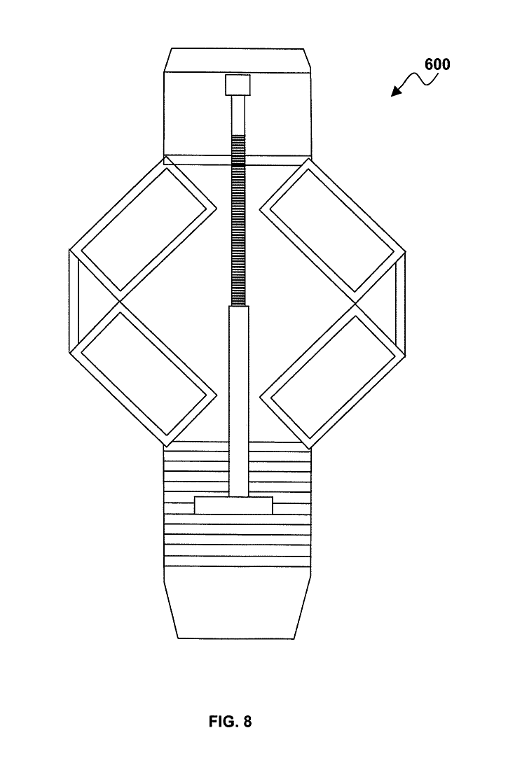

FIG. 6 illustrates a perspective view of an implant cage 600, in accordance with an alternative embodiment. The cage expansion mechanism 150 includes expandable compartments 601, 602, 603, 604, and 605. Upon tightening the male and female screw arrangement 105, the compartments 601, 602, 603, 604, and 605 stretches and increases the size of the cage 600. FIG. 7 illustrates a top view of implant cage 600 of FIG. 6, in accordance an alternative embodiment. FIG. 8 illustrates a perspective view of implant cage 600 of FIG. 6 after expansion, in accordance with an alternative embodiment. FIG. 9 illustrates a perspective view of vertebral endplate 700 with the implant cage 600 of FIG. 8, in accordance with an alternative embodiment.

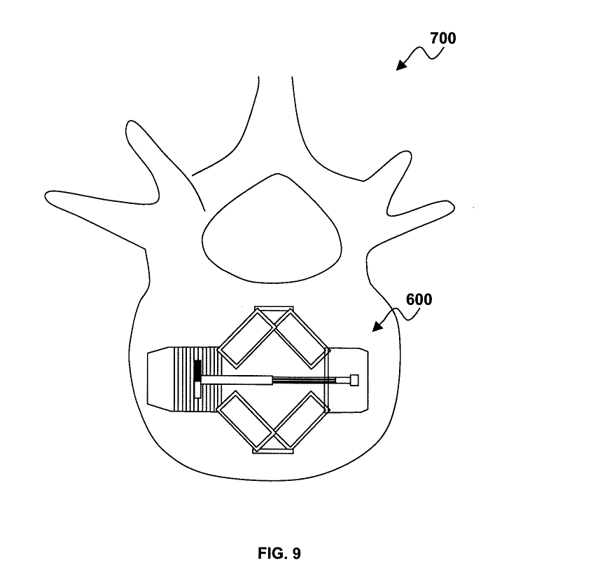

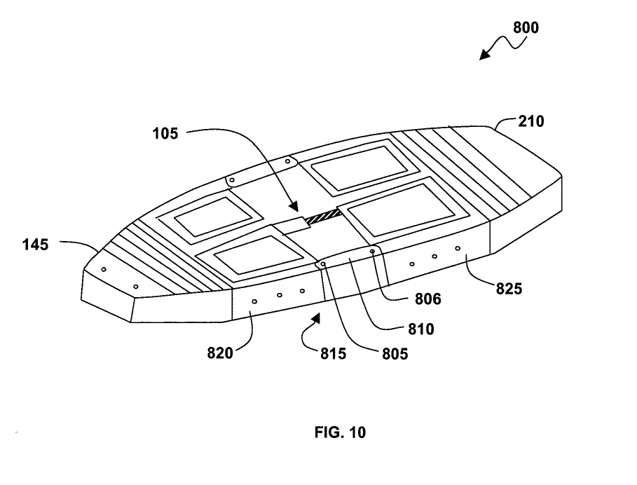

FIG. 10 illustrates a perspective view of an implant cage 800, in accordance with an alternative embodiment. The expansion mechanism 815 includes pins 805 and 806, hinge 810, and a set of compartments 820 and 825. The expansion mechanism 815 stretches and increases the cage size upon tightening the male and female screw arrangement 105. FIG. 11 illustrates a perspective view of vertebral endplate 900 with the implant cage 800 of FIG. 10, in accordance with an alternative embodiment.

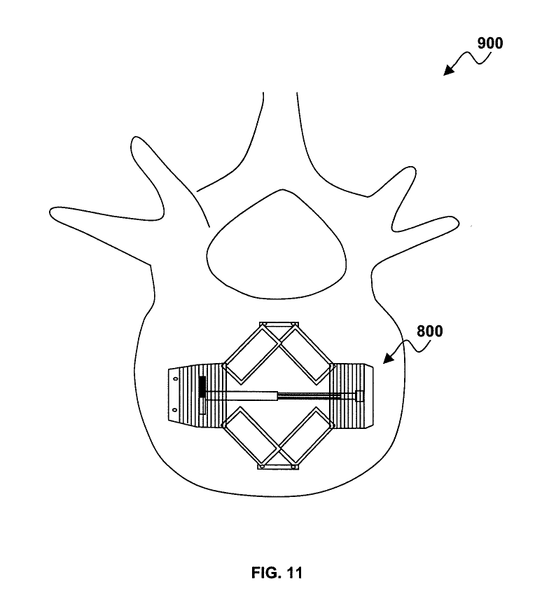

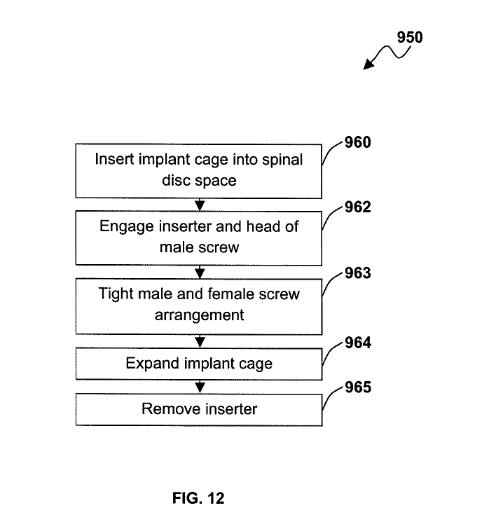

FIG. 12 illustrates a high-level flow chart 950 depicting implantation process for spinal fusion from lateral approach, in accordance with the disclosed embodiments. As illustrated at block 960, the inserter is utilized for inserting the cage into the spinal disc space using lateral approach. Then, the inserter is engaged with the male screw head as indicated at block 962. As depicted at block 963, the male and female screw arrangement can be tightened utilizing the inserter discussed herein. According to the required space between the endplates of upper and lower vertebra, the cage can be expanded as illustrated at block 964. Finally, the inserter is removed as described at block 965.

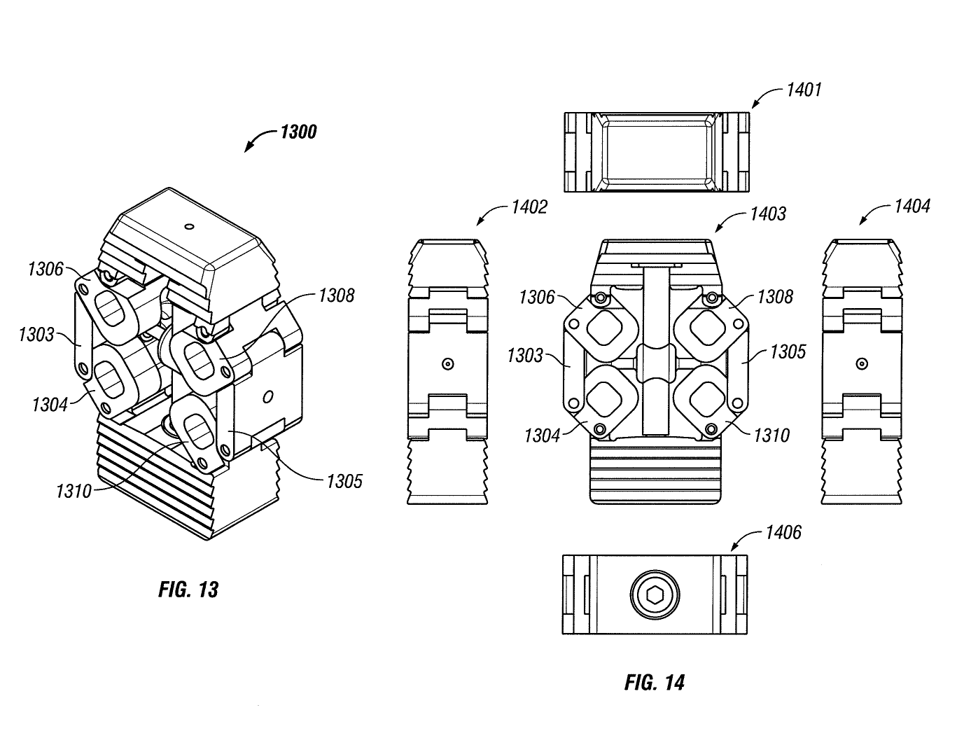

FIG. 13 illustrates a perspective view of an implant apparatus 1300 for spinal fusion, in accordance with another embodiment. As shown in FIG. 13, moveable sections 1304, 1306, 1308, and 1310 are illustrated. Section 1303 links section 1304 and 1306 to one another, while section 1305 links sections 1308 and 1310 in the configuration shown in FIG. 13.

FIG. 14 illustrates a left side view 1402, a front view 1403, a right side view 1404, a bottom view 1406, and a top view 1401 of the implant cage of the apparatus 1300 depicted in FIG. 13. Note that in FIGS. 13-14, similar or like parts are general indicated by identical reference numerals.

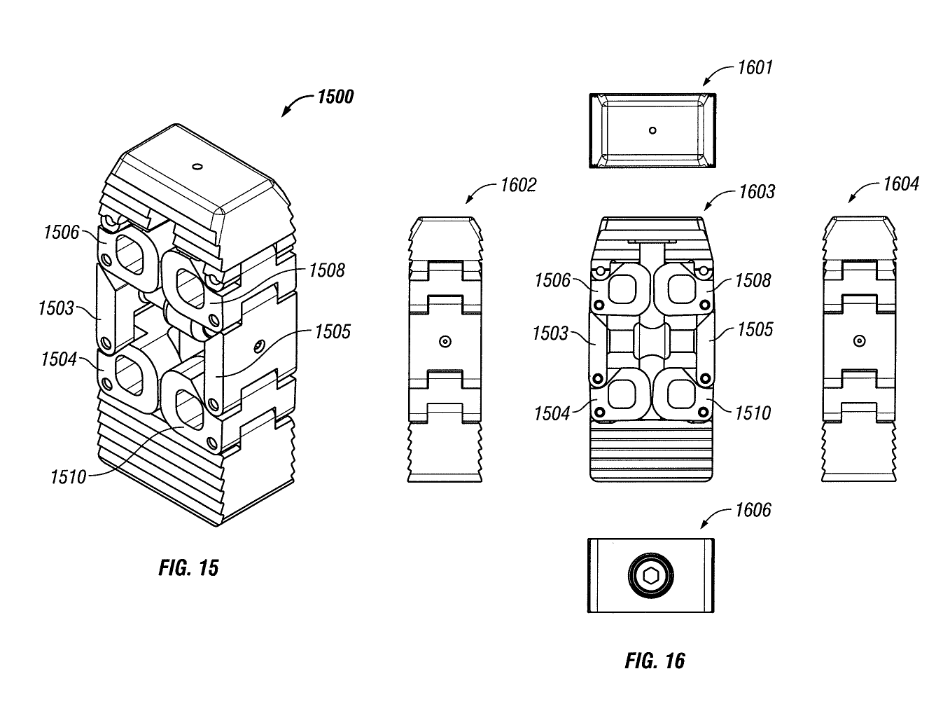

FIG. 15 illustrates a perspective view of an implant apparatus 1500 for spinal fusion, in accordance with another embodiment. As shown in FIG. 15, moveable sections 1504 and 1506 connect to section 1503, and sections 1508 and 1510 are linked via section 1505.

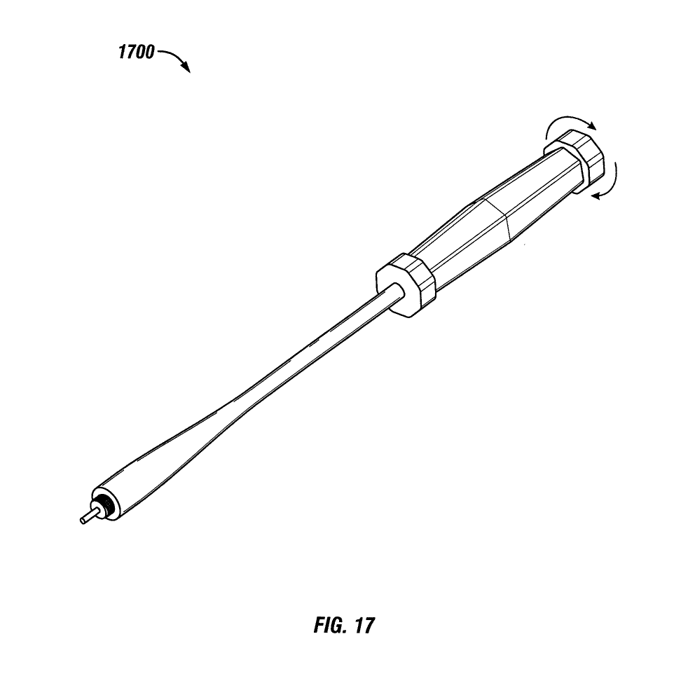

FIG. 16 illustrates a left side view 1602, a front view 1603, a right side view 1604, a bottom view 1606, and a top view 1601 of the implant cage of the apparatus depicted in FIG. 15. FIG. 17 illustrates a perspective view of an inserter 1700 that can be utilized in accordance with the disclosed embodiments. The inserter 1700 shown in FIG. 17 thus represents an alternative embodiment (e.g. a variation to inserter 400) for use in spinal implantation processes.

Based on the foregoing, it can be appreciated that an implant apparatus and a method for spinal fusion from oblique, lateral, ALIF, PLIF, and/or TLIF approaches are disclosed. The apparatus/device can be configured to include an expandable implant cage and an inserter. The cage can be inserted between endplates of upper and lower vertebra using oblique, lateral, ALIF, PLIF, and TLIF approach. The cage generally includes a male and female screw configuration and a cage expansion mechanism. The inserter inserts the cage in a spinal disc space and tightens the male and female screw arrangement. Once the cage is inserted to the desired position, viewed by X-ray you will begin to tighten the male portion of the screw in the device, and continue to tighten until final deployment of cage has been achieved. This provides a much greater footprint that allows the device to reach the cortical ring or apophyseal ring of the vertebral body. Tightening of male and female screw arrangement operates the cage expansion mechanism to expand the cage size. The cage can be inserted through a smaller surgical opening and then expanded to a full size assembly between the vertebrae.

Based on the foregoing, it can be appreciated that various embodiments are disclosed, including preferred and alternative embodiments. For example, in an embodiment, an implant apparatus for spinal fusion can include an expandable implant cage positioned between endplates of upper and lower vertebra comprising a male and female screw arrangement and a cage expansion mechanism, wherein the cage expansion mechanism expands the cage size on tightening the male and female screw arrangement; and an inserter for inserting the cage in a spinal disc space that maintains a handle, a shaft, and a coupling arrangement, wherein the inserter is operated to engage the coupling arrangement with the male and female screw arrangement and to tighten the male and female screw arrangement.

In some embodiments, the cage expansion mechanism can comprise a pin and hinge configuration. In other embodiments, the cage expansion mechanism can comprise a cage compartment configuration. In yet other embodiments, the cage expansion mechanism can comprise a combination of a cage compartment configuration and a pin and hinge configuration. In still other embodiments, the disclosed cage expansion mechanism can be positioned on at least one sidewall of the cage. In some embodiments, the disclosed spinal fusion can be an oblique approach. In other embodiments, the spinal fusion can be a lateral approach. In yet other embodiments, the spinal fusion can be an ALIF approach, a PLIF approach, OF a TLIF approach.

In another embodiment, an implant apparatus for spinal fusion can include, for example, an expandable implant cage positioned between endplates of upper and lower vertebra comprising a male and female screw arrangement and a cage expansion mechanism, wherein the cage expansion mechanism expands the cage size on tightening the male and female screw arrangement. Such an apparatus can also include an inserter for inserting the cage in a spinal disc space that maintains a handle, a shaft, and a coupling arrangement, wherein the inserter is operated to engage the coupling arrangement with the male and female screw arrangement and to tighten the male and female screw arrangement. Additionally, in such an apparatus, the cage expansion mechanism can comprise at least one of: a pin and hinge configuration, a cage compartment configuration, or a combination of the cage compartment configuration and the pin and hinge configuration. In an alternative embodiment of such an apparatus, the cage expansion mechanism can be positioned on at least one sidewall of the cage. In yet another embodiment of such an apparatus, the spinal fusion can be, for example, a lateral approach, an ALIF approach, a PLIF approach, a TLIF approach, and/or an oblique approach.

In still another embodiment, a method for spinal fusion may be implemented, which includes, for example, the steps of locating an expandable implant cage between endplates of upper and lower vertebra comprising a male and female screw arrangement and a cage expansion mechanism, wherein the cage expansion mechanism expands the cage size on tightening the male and female screw arrangement; providing an inserter for inserting the cage in a spinal disc space that maintains a handle, a shaft, and a coupling arrangement; and operating the inserter to engage the coupling arrangement with the male and female screw arrangement and to tighten the male and female screw arrangement.

In another embodiment of such a method, a step may be implemented for positioning the cage expansion mechanism on at least one sidewall of the cage. In other embodiments of such a method, a step may be implemented for configuring the cage expansion mechanism to comprise at least one of: a pin and hinge configuration; a cage compartment configuration; or a combination of the cage compartment configuration and the pin and hinge configuration.

It will be appreciated that variations of the above disclosed and other features and functions, or alternatives thereof, may be desirably combined into many other different systems or applications. Also, that various presently unforeseen or unanticipated alternatives, modifications, variations or improvements therein may be subsequently made by those skilled in the art which are also intended to be encompassed by the following claims.

What is claimed is:

1. An implant apparatus for spinal fusion, said apparatus comprising:
an expandable implant cage positioned between endplates of upper and lower vertebra comprising a male and female screw arrangement and a cage expansion mechanism, wherein said cage expansion mechanism expands said cage size on tightening said male and female screw arrangement; and
an inserter for inserting said cage in a spinal disc space that maintains a handle, a shaft, and a coupling arrangement, wherein said inserter is operated to engage said coupling arrangement with said male and female screw arrangement and to tighten said male and female screw arrangement.

2. The apparatus of claim 1 wherein said cage expansion mechanism comprises a pin and hinge configuration.

3. The apparatus of claim 1 wherein said cage expansion mechanism comprises a cage compartment configuration.

4. The apparatus of claim 1 wherein said cage expansion mechanism comprises a combination of a cage compartment configuration and a pin and hinge configuration.

5. The apparatus of claim 2 wherein said cage expansion mechanism is positioned on at least one sidewall of said cage.

6. The apparatus of claim 3 wherein said cage expansion mechanism is positioned on at least one sidewall of said cage.

7. The apparatus of claim 4 wherein said cage expansion mechanism is positioned on at least one sidewall of said cage.

8. The apparatus of claim 1 wherein said spinal fusion comprises an oblique approach.

9. The apparatus of claim 1 wherein said spinal fusion comprises a lateral approach.

10. The apparatus of claim 1 wherein said spinal fusion comprises an ALIF approach.

11. The apparatus of claim 1 wherein said spinal fusion comprises a PLIF approach.

12. The apparatus of claim 1 wherein spinal fusion comprises a TLIF approach.

13. An implant apparatus for spinal fusion, said apparatus comprising:
an expandable implant cage positioned between endplates of upper and lower vertebra comprising a male and female screw arrangement and a cage expansion mechanism, wherein said cage expansion mechanism expands said cage size on tightening said male and female screw arrangement;
an inserter for inserting said cage in a spinal disc space that maintains a handle, a shaft, and a coupling arrangement, wherein said inserter is operated to engage said coupling arrangement with said male and female screw arrangement and to tighten said male and female screw arrangement; and
wherein said cage expansion mechanism comprises at least one of:
a pin and hinge configuration;
a cage compartment configuration; or
a combination of said cage compartment configuration and said pin and hinge configuration.

14. The apparatus of claim 13 wherein said cage expansion mechanism is positioned on at least one sidewall of said cage.

15. The apparatus of claim 13 wherein said spinal fusion comprises a lateral approach.

16. The apparatus of claim 13 wherein said spinal fusion comprises an ALIF approach.

17. The apparatus of claim 13 wherein said spinal fusion comprises a PLIF approach.

18. The apparatus of claim 13 wherein said spinal fusion comprises a TLIF approach.

19. The apparatus of claim 13 wherein said spinal fusion comprises an oblique approach.

20. A method for spinal fusion, said method comprising:
locating an expandable implant cage between endplates of upper and lower vertebra comprising a male and female screw arrangement and a cage expansion mechanism, wherein said cage expansion mechanism expands said cage size on tightening said male and female screw arrangement;
providing an inserter for inserting said cage in a spinal disc space that maintains a handle, a shaft, and a coupling arrangement; and
operating said inserter to engage said coupling arrangement with said male and female screw arrangement and to tighten said male and female screw arrangement.

21. The method of claim 20 further comprising positioning said cage expansion mechanism on at least one sidewall of said cage.

22. The method of claim 20 further comprising configuring said cage expansion mechanism to comprise at least one of:
a pin and hinge configuration;
a cage compartment configuration; or
a combination of said cage compartment configuration and said pin and hinge configuration.
